(19) United States
(12) Patent Application Publication
(10) Pub. No.: US 2005/0273087 A1
(43) Pub. Date: Dec. 8, 2005

(54) Catheter guide wire insertion tool

(76) Inventors: Russ Houser, Livermore, CA (US); Christopher J. White, New Orleans, LA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/198,554

(22) Filed: Aug. 5, 2005

Related U.S. Application Data
(63) Continuation of application No. 10/272,209, filed on Oct. 15, 2002.
(60) Provisional application No. 60/343,627, filed on Oct. 22, 2001.

(51) Int. Cl. A61B 17/00

(52) U.S. Cl. 606/1

(57) ABSTRACT

A device for introducing a guide wire into a catheter comprising a housing having at least one lumen for permitting the passage of a guide wire therethrough. The lumen has an axial single seam positioned longitudinally along the lumen that is defined by opposite lateral ends of the housing. The seam comprises a gap that is dimensioned less than the diameter of the guide wire to be passed therethrough for preventing lateral removal of the guide wire from said device during use. The housing further comprises a plurality of gripping surfaces distinct from the seam for permitting a user to widen the seam to facilitate removal of a guide wire. The housing may further include an axial notch positioned generally about 180 degrees opposite from the seam to facilitate widening of the seam when it desired to remove the guide wire. The join may comprise a longitudinal groove on the housing. The gripable surfaces may each comprise a tab, or may comprise an indentation in the housing.

### RELATED APPLICATIONS

The present application is a continuation of U.S. application Ser. No. 10/272,209 filed Oct. 15, 2002, which claims priority 35 U.S.C. §119(e) to U.S. Provisional Application No. 60/343,627 filed Oct. 22, 2001, the disclosures of which are incorporated by reference herein in their entirety.

### FIELD OF THE INVENTION

The present invention relates to a device for facilitating the insertion of a guide wire into a catheter lumen in a manner that the device can be removed from the guide wire while the guide wire remains loaded into the catheter.

### BACKGROUND OF THE INVENTION

In the treatment of human diseases and disorders, clinicians and interventionalists often routinely use some form of catheter based treatment system, whether for the aspiration or infusion of fluids, or for more elaborate procedures such as angioplasty. To advance a catheter or other tubular member within a patient's vasculature or other systemic lumen, it is often desired to use a guide wire pre-inserted into the patient to first locate and then preserve the pathway for the catheter. The catheter is advanced over the guide wire to a desired distal location, at which point the guide wire may be left in place or removed.

A difficulty encountered in the use of a guide wire and catheter treatment system is inserting the guide wire through the lumen of the catheter. The internal lumen of catheters used in many different applications tends to be rather small, as small as 0.010 inches. In contrast, guide wires tend to have a diameter only a few thousands of an inch smaller than the intended lumen. Coupled with the fact that the distal end of a catheter is often tapered, the insertion of the proximal end of the guide wire into the distal end of the catheter or other tubular member is difficult at best. The resiliency of the catheter material adds to the difficulty. In the circumstances of medical intervention, such as a catheterization lab, where the lighting may be poor, intentionally or unintentionally, the difficulty of guide wire insertion is exacerbated.

There have been efforts made to address this difficulty. For example, U.S. Pat. No. 5,320,613 to Houge et al. discloses a device that facilitates insertion of a guide wire into a catheter lumen. However, the Houge et al. device is flawed in that a longitudinal groove or slot is provided along the length of the device that has a width that is greater than the guide wire to permit the lateral removal of the guide wire from the device. The slot requires that the clinician hold his/her thumb over the slot to permit the guide wire insertion process to proceed. An alternative embodiment disclosed by Houge et al. adds a rotatable cover sleeve that covers the slot during use and, when rotated to expose the slot, permits removal of the guide wire after use.

U.S. Pat. No. 5,978,699 to Fehse et al. also discloses a device that facilitates insertion of a guide wire into a catheter lumen. However, the Fehse et al. device is also flawed in that it presents a large cumbersome configuration that relies upon a hinged format to create a longitudinal slot through which the guide wire may be removed after use.

The present invention reflects an improvement over the prior art by providing a self-contained guide wire insertion tool that is streamlined in configuration and avoids the need for an additional cover sleeve or the need for the clinician to hold his/her thumb over a removal slot.

### SUMMARY OF THE INVENTION

The present invention comprises a device for introducing a guide wire into a catheter, where the device comprises a housing having at least one lumen for permitting the passage of a guide wire therethrough. The lumen has an axial single seam positioned longitudinally along the lumen that is defined by opposite lateral ends of the housing. The seam comprises a gap that is dimensioned less than the diameter of the guide wire to be passed therethrough for preventing lateral removal of the guide wire from said device during use. The housing further comprises a plurality of gripping surfaces distinct from the seam for permitting a user to widen the seam to facilitate removal of a guide wire. The housing may further include an axial notch positioned generally about 180 degrees opposite from the seam to facilitate widening of the seam when it desired to remove the guide wire. The join may comprise a longitudinal groove on the housing. The gripable surfaces may each comprise a tab, or may comprise an indentation in the housing.

### BRIEF DESCRIPTION OF THE DRAWINGS

FIG. 1 is a side schematic view of the present invention insertion tool.

FIG. 2 is an end schematic view of the device of FIG. 1 taken along lines 2-2.

FIG. 3 is a side cross-sectional schematic view of the device of FIG. 1 taken along lines 3-3.

FIG. 4 is a schematic view of the present invention insertion tool engaging a catheter through which it is desired to advance a guide wire.

FIG. 5 is a cross-section schematic of the arrangement shown in FIG. 4.

### DETAILED DESCRIPTION OF THE INVENTION

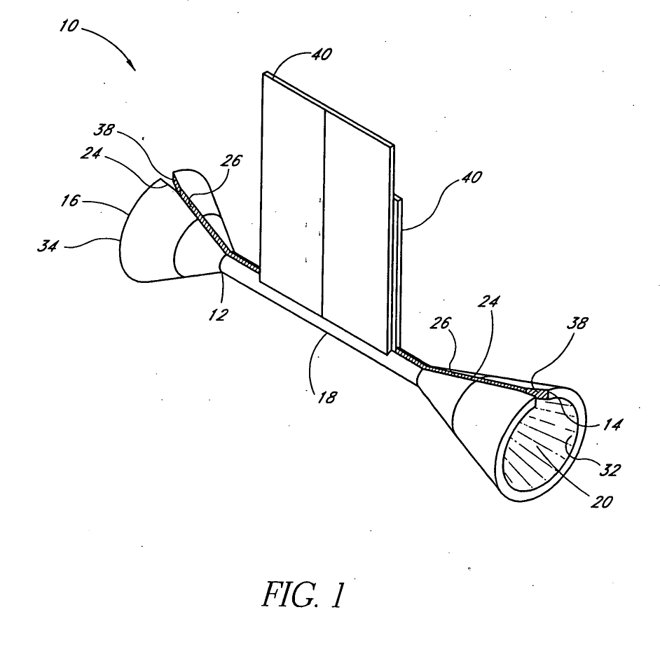

Referring to FIGS. 1-3, the invention comprises a guide wire insertion tool 10 that itself comprises a housing 12 having a proximal end 14, a distal end 16, and a mid-section 18 therebetween. Traversing longitudinally through the housing is a lumen 20 of sufficient diameter to permit the axial passage of a guide wire therethrough. Either of the proximal or distal end may be fitted with a Luer fitting (not shown) for engagement with a mating Luer fitting of a discrete piece. The insertion tool also includes a means for separating the insertion tool from a guide wire after use. In a first embodiment, the housing is configured in a rolled format so as to comprise one configuration of separating means: two lateral sides 24 and 26 spaced apart to define an already exposed seam 30. The seam 30 of this first embodiment comprises a gap that is sufficiently small in its normal position to preclude the unintended removal of the guide wire from the housing during use.

The housing 12 is preferably tapered radially outward from the mid-section 18 toward the proximal end 14 and distal end 16, respectively so as to provide a funnel-like appearance. The large diameter ends define proximal and distal openings 32 and 34 that serve to permit easy feeding of a guide wire into the lumen 20. Like the profile of the housing, the profile of the lumen 20 preferably has a taper extending radially outward from the mid-section 18 toward the proximal end 14 and distal end 16. Having a tapered lumen facilitates effective engagement of the insertion tool 10 with a catheter during use, as explained more fully below. The interior surface of the lumen 20 is preferably smooth and untextured, although it is not necessary for effective operation. Within the mid-section, the lumen preferably has a diameter only slightly larger than the diameter of the guide wire to restrict undesired lateral movement of the guide wire, which permits greater control during advancement of the guide wire through the insertion tool. In one embodiment, for guide wires having diameters approximately 0.014 inches, it is contemplated that the interior diameter of lumen 20 within mid-section 18 would be approximately 0.017 inches, although other diameters would be effective.

In the preferred embodiment, each of the proximal end 14 and distal end 16 includes a recess 38 that serves the purpose of directing the guide wire through seam 30 when it is desired to remove the guide wire from the insertion tool, although such a recess is not necessary for effective use. When removing the guide wire insertion tool 10 from a guide wire, the tool 10 may be angled in such a way that the guide wire is directed to one of the recesses 38 on the tool. Once the guide wire is at the notched location, the tool may continue to be angled in such a way that the guide wire is urged through seam 30. In this manner, the tool may be removed from around the guide wire, using only tactile feel, without looking at the guide wire or tool.

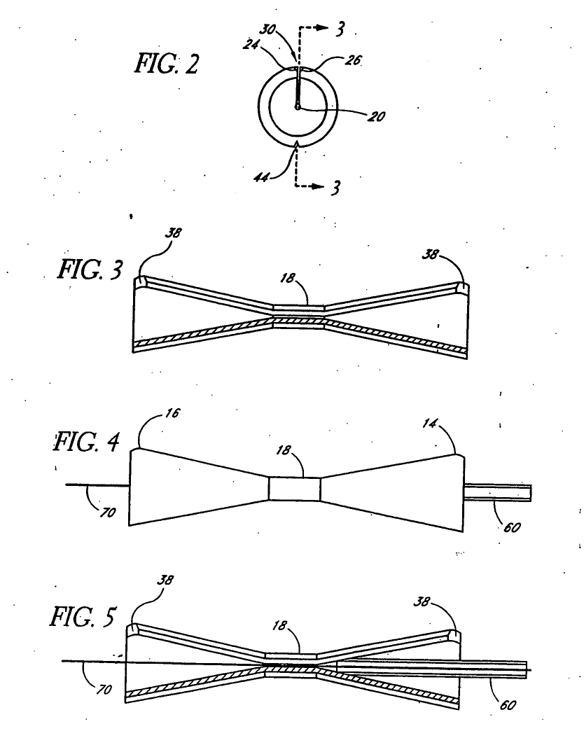

The separating means of housing 12 further comprises means for gripping the housing in a manner that permits widening the seam 30 to permit separation of the guide wire form the insertion tool 10. In one embodiment, shown in FIGS. 1 and 2, the gripping means comprises separator tabs 40 extending radially outward from the housing surface. The tabs 40 are joined to the housing 12 at opposite sides of the seam 30 to permit a user to grip the tabs 40 and pull them apart. Doing so results in the seam gap widening sufficiently to permit removal of the guide wire. The tabs may be positioned directly across from each other, or may be positioned longitudinally offset from each other. In the former case, the separator tabs 40 should be spaced apart to permit the user to effectively grasp the tabs. Other arrangements for the tab are also contemplated, so long as they permit the user to grip the tabs and widen the seam 30 for guide wire separation. The tabs 40 may be made integral with the housing or may be discrete components attached to the housing. In another embodiment of the separating means, the gripping means may comprise indentations in the housing at the already exposed seam that permit the user to grip opposing lateral sides 24 and 26 of the housing to widen the seam gap.

The insertion tool may optionally further comprise means for facilitating widening of the seam 30. In the preferred embodiment, the facilitating means comprises a longitudinally extending notch 44, as shown more clearly in FIG. 2. Given housing material that is sufficiently pliable, the notch 44 facilitates widening of the housing seam 30 by reducing the thickness of the housing wall at a point radially opposite of the seam 30. That reduced wall thickness results in a weaker resistance to radial bending of the housing 12, thus facilitating widening of the seam 30. In effect, the notch 44 presents a living hinge-like arrangement. In an alternative embodiment, the housing material is sufficiently rigid that the housing intentionally breaks at the notch. In that embodiment, widening of the seam is not the goal. Other configurations for the facilitating means are also contemplated, including longitudinal scoring that results in a weakened housing portion, and other means known to persons of ordinary skill in the art. Indeed, no notch or scoring is necessary if it is desired nearly to have the facilitating means comprise a thinner wall that permits resilient bending of the wall at the thinner wall area.

As described above, the already exposed seam 30 through which the guide wire may be separated from the insertion tool 10 is preferably defined by opposing lateral sides of the housing 12. Other means for separating the guide wire from the housing are contemplated. For example, instead of an exposed seam, the housing may comprise a longitudinally arranged set of perforations that permit the user to break the housing wall at the perforations for guide wire separation. Such an arrangement would further comprise gripping means that may be similar to that described above, or some other effective gripping means. Yet another alternative embodiment of separating means comprises a tearable housing wall portion that would permit a user, by gripping a tab or other like grabable protrusion, to remove a longitudinal section of the housing wall from proximal end to distal end (or vice versa), thereby exposing a seam for separation of the guide wire from the insertion tool. No gripping means for widening the seam would be necessary with such an arrangement. With at least some of these alternative arrangements, means for facilitating widening of the seam may be used if so desired.

Referring to FIGS. 4 and 5, the present invention also comprises one or more methods of using the inventive insertion tool 10 in which one method comprises the step inserting the distal end of a catheter 60 into the proximal end 14 of the housing 12 to permit fairly tight engagement of the catheter tip with the interior tapered portion of lumen 20. Often distal tips of catheters are themselves tapered, permitting more effective engagement with the tapered portion of lumen 20, as shown more clearly in FIG. 5. The method further comprises the steps of inserting a guide wire 70 into the distal end 16 of the housing and advancing the guide wire through the lumen 20 into the mid-section 18. Continued advancement of the guide wire 70 results in the guide wire 70 being smoothly directed into the distal end of catheter 60. Once the guide wire 70 is sufficiently advanced within the catheter, the insertion tool 10 is no longer needed and may be separated from the guide wire following the inventive steps. With one embodiment described herein, the method further comprises gripping the gripping means, in one case tabs 40, to widen the seam 30 and permit removal of the guide wire from lumen 20. Once the insertion tool 10 is separated, the clinician may then proceed with further treatment of the patient by advancing the catheter 60 along the guide wire 70 into the patient. Alternative methods comprise the alternative step of using an insertion tool that employs indentations rather than gripping tabs, that permit a user to grip the housing in such a away as to widen the seam. Alternative steps include gripping a pull tab longitudinally along the housing to expose a seam that is sufficiently wide to permit separation of the guide wire.

It is contemplated that the present inventive insertion tool 10 be made of material that is sufficiently rigid and stable to permit insertion and advancement of a guide wire into the housing 12, yet sufficiently resilient to permit opening of the seam to remove the guide wire. In one preferred embodiment, the housing 12 is made of a thermoplastic rubber (TPR) such as that sold under the brand name Santoprene®, for example. Other medical grade materials may also be used as well, including metals, metal alloys, silicone, polymers such as polycarbonate, polyethylene, polyester, polypropylene, polyurethane, fluoropolymers, PVC or other polymers, including those sold under the trade names Pebax® and Surlyn®. The contemplated hardness is preferably approximately 70-100 on the Shore A scale and approximately 20-50 on the Shore D scale. It should be recognized that materials having a finished hardness outside this range would also be effective for the application described herein. The insertion device of the present invention may incorporate a biocompatible fluorescing feature, making the device easier to see in the clinical setting. The florescence may be realized by using a coating, or by including fluorescing materials during the compounding or molding (or other) manufacturing processes. Also, the external surface of the housing may be textured to provide better gripping by the clinician.

Contemplated methods of manufacture include injection molding, casting, machining, extrusion, a combination of any of these, or some other suitable manufacturing method. The insertion tool may be made of unitary construction, or a combination of assembled components.

One anticipated advantage of the present inventive insertion tool is that is may be reusable during multiple catheter insertions for a single patient. By employing a living hinge in the design, i.e., the longitudinal notch that permits the seam to be widened without breaking the housing, the present invention is reusable, provided adequate sterilization procedures are applied. Being reusable for a single patient reduces the incremental cost per procedure and further increases the speed and ease of use. Since multiple wire exchanges are done on a single patient, being able to reuse cuts down on the incremental time to open additional packages associated with incremental wire exchanges.

It should be noted that the present invention insertion tool may also be used outside the clinical context for the feeding of any thin object into a correspondingly thin opening. It is also contemplated that a set of insertion tools may be provided, each with a different diameter lumen for guiding differently sized objects therethrough, where removal of the tool from around the side is useful.

1. A reusable insertion tool device for introducing a guide wire into a catheter, the device, said device configured to permit repeated separation of the guide wire from the device, said device comprising a housing having:
a lumen therethrough to temporarily house a guidewire,
an exposed longitudinal seam, and
a longitudinal notch facilitating the widening of the seam when desired to remove a guide wire therethrough.

2. The device of claim 1, wherein the width of the seam is smaller than about 0.14 inches, the diameter of a conventional guide wire, such that the guide wire cannot be removed from the lumen through the longitudinal seam until the seam has been widened.

3. The device of claim 1, wherein the device further comprises gripping means for facilitating widening of the seam.

4. The device of claim 3, wherein the gripping means comprises tabs.

5. The device of claim 3, wherein the gripping means comprises indentations in the housing.

6. The device of claim 1 wherein the seam is created by pulling a tab on the housing to remove a portion of the housing wall.

7. A device for introducing a guide wire into a catheter, comprising a generally tubular housing comprising a first converging section, a second cylindrical section, and a third diverging section, the housing having a two ends and a lumen therebetween, said housing further having longitudinal seam along the housing, whereby the housing has a generally C-shaped cross-section, said seam defined by opposite lateral edges of said generally C-shaped housing, said housing further having means for permitting a user to expand the seam to permit the withdrawal of a guide wire therefrom.

8. The device of claim 7, wherein the device comprises essentially a one-piece tube.

9. The device of claim 7, wherein the housing further comprises protrusions extending radially outward from the housing for gripping the housing in a manner that permits the protrusions to be pulled apart and, thus, expand the seam for the removal of the guide wire.

10. The device of claim 7, further comprising a longitudinal weakened area of the housing, oriented parallel to, and located generally opposite to, said seam to facilitate widening of the seam.

11. The device of claim 10, wherein the weakened area comprises a longitudinal notch on the housing.
